(19) United States
(12) Patent Application Publication
(10) Pub. No.: US 2022/0112468 A1
(43) Pub. Date: Apr. 14, 2022

(54) NOVEL REPROGRAMMING METHOD

(71) Applicant: Babraham Institute, Cambridge (GB)

(72) Inventors: Wolf REIK, Cambridge (GB); Diljeet GILL, Cambridge (GB); Thomas STUBBS, Cambridge (GB)

(21) Appl. No.: 17/555,793

(22) Filed: Dec. 20, 2021

Related U.S. Application Data
(63) Continuation of application No. PCT/GB2020/051668, filed on Jul. 10, 2020.
(60) Provisional application No. 62/872,910, filed on Jul. 11, 2019.

(30) Foreign Application Priority Data
Jul. 11, 2019 (GB) 1909975.3

(51) Int. Cl. C12N 5/074; C12N 15/86; C12N 15/90; C12Q 1/6883; G01N 33/50; C12Q 1/6809; A61K 35/545; A61K 8/98; A61Q 19/08

(52) U.S. Cl. CPC C12N 5/0696; C12Q 2600/154; C12N 15/907; C12Q 1/6883; G01N 33/502; G01N 33/5073; C12Q 1/6809; A61K 35/545; A61K 8/981; A61Q 19/08; C12N 2501/603; C12N 2501/602; C12N 2501/604; C12N 2501/606; C12N 2506/1307; C12N 2740/15043; C12N 2830/003; C12N 2800/80; C12N 15/86

(57) ABSTRACT

The invention relates to methods of reprogramming a somatic cell comprising culturing the somatic cell in the presence of one or more Yamanaka factors and further culturing said somatic cell in the absence of said one or more Yamanaka factors. The invention further relates to a reprogrammed somatic cell produced according to the methods as defined herein. Also provided are cosmetic methods, cosmetic compositions, a reprogrammed somatic cell and compositions for use in treatment or rejuvenation, as well as methods for screening age modulating agents, factors and/or cellular processes, comprising the methods and a reprogrammed somatic cell as defined herein.

### FIELD OF THE INVENTION

The invention relates to methods of reprogramming a somatic cell comprising culturing the somatic cell in the presence of one or more Yamanaka factors and further culturing said somatic cell in the absence of said one or more Yamanaka factors. The invention further relates to a reprogrammed somatic cell produced according to the methods as defined herein. Also provided are cosmetic methods, cosmetic compositions, a reprogrammed somatic cell and compositions for use in treatment or rejuvenation, as well as methods for screening age modulating agents, factors and/or cellular processes, comprising the methods and a reprogrammed somatic cell as defined herein.

### BACKGROUND OF THE INVENTION

Ageing is characterised by a gradual loss of function occurring at the molecular, cellular, tissue and organismal levels. As we age, the pattern of DNA methylation at the chromatin level changes with some sites gaining and some sites losing this mark. DNA methylation is an epigenetic modification that plays many roles in mammalian cells ranging from transposable element silencing to X chromosome inactivation and, as such, changes and progressive accumulation of epigenetic marks are associated with aberrant gene expression and regulation, stem cell exhaustion, senescence and dysregulated tissue homeostasis. These changes are relatively consistent between individuals and can be used to predict age. Such predictors (e.g. the Horvath epigenetic clock) produce a value called DNA methylation age (also known as epigenetic age), which is thought to represent the biological age of an individual or a tissue. Lifestyle factors that affect the ageing process (e.g. diet) can also affect DNA methylation age. Nevertheless, the biology underlying the epigenetic clock and the DNA methylation age remains unclear.

During the process of induced pluripotent stem (iPS) cell reprogramming, somatic cells are converted or de-differentiated into pluripotent stem cells. Gene expression profiling has revealed 3 phases of reprograming: initiation, maturation and stabilisation. While the initiation phase is characterised by an immediate mesenchymal-to-epithelial transition, the expression of a subset of pluripotency-associated genes (OCT4, NANOG and SALL4) is detected in the maturation phase. Acquisition of the final iPS cell state requires a late stabilisation phase marked by the expression of the remaining pluripotency-associated genes (such as UTF1, LIN28, DPPA2 and DPPA4). The resulting iPS cells are similar to natural pluripotent stem cells (e.g. embryonic stem (ES) cells) in many aspects, including in their ability to differentiate into multiple cell types. However, during iPS cell reprogramming, DNA methylation age is reset to zero years old regardless of the age of the donor tissue from which the somatic cell was obtained. As such, the process of iPS cell reprogramming resets the epigenetic signature of the somatic cell to an embryonic-like state and causes loss of somatic cell lineage identity.

There is therefore a need to produce reprogrammed somatic cells which have a reduced DNA methylation age or epigenetic age but retain their lineage identity. Such reprogrammed cells will find use in numerous therapeutic and cosmetic applications as well as in the treatment and/or amelioration of age-related or degenerative diseases and disorders.

### SUMMARY OF THE INVENTION

According to a first aspect of the invention, there is provided a method of reprogramming a somatic cell to a pluripotent-like or rejuvenated state, comprising:
i) culturing said somatic cell in the presence of one or more Yamanaka factors for a period of at least 5 days, and/or until expression of a pluripotency marker is detectable on the surface of or within said somatic cell, and/or until a somatic cell lineage-specific marker is no longer detectable on the surface of said somatic cell;
ii) further culturing said somatic cell in the absence of said one or more Yamanaka factors until expression of said pluripotency marker has reduced on the surface of or within said somatic cell, and/or until expression of a somatic cell lineage-specific marker is detected on the surface of said somatic cell.

According to a further aspect of the invention, there is provided a reprogrammed somatic cell produced according to the methods as defined herein.

According to another aspect of the invention, there is provided a pharmaceutical composition comprising a reprogrammed somatic cell as defined herein.

According to a yet further aspect of the invention, there is provided a reprogrammed somatic cell as defined herein or a pharmaceutical composition as defined herein for use in the treatment and/or amelioration of a degenerative or age-related disease or disorder or for use in the rejuvenation of a tissue or organ.

According to one further aspect, there is provided a cosmetic composition comprising a reprogrammed somatic cell as defined herein.

According to a yet further aspect, there is provided a cosmetic method of regenerating or rejuvenating skin comprising administration or application of a reprogrammed somatic cell as defined herein or a cosmetic composition as defined herein to a subject in need thereof.

According to a further aspect, there is provided a method of screening for an age modulating agent, said method comprising:
(i) performing the method as defined herein in the presence and the absence of a test agent to generate a reprogrammed somatic cell; and
(ii) determining the molecular signature, such as the epigenetic signature, of the reprogrammed somatic cell,
   wherein a difference between the molecular signature determined for a reprogrammed somatic cell generated in the presence of the test agent and the molecular signature determined for a reprogrammed somatic cell generated in the absence of the test agent is indicative of the age modulating effect of said agent.

According to a yet further aspect, there is provided a method of screening for an age modulating factor or cellular process, said method comprising:
(i) reprogramming a somatic cell from a diseased tissue or organ according to the method as defined herein; and
(ii) determining the molecular signature, such as the epigenetic signature, of the reprogrammed somatic cell from a diseased tissue or organ and of a reprogrammed somatic cell as defined herein or of a non-reprogrammed somatic cell from said diseased tissue or organ,
   wherein a difference between the molecular signature determined for the reprogrammed somatic cell from a diseased tissue or organ and the molecular signature determined for the reprogrammed somatic cell as defined herein or the non-reprogrammed somatic cell from the diseased tissue or organ is indicative of the age modulating factor or cellular process associated with the disease.

### BRIEF DESCRIPTION OF THE FIGURES

FIG. 1: Flow cytometric plots showing surface expression of CD13 and SSEA4 on human somatic fibroblast cells after 13 days of culture with expression of Yamanaka factors (plots labelled with “+”). Negative control cultures did not express the Yamanaka factors (lower plots; labelled with “−”).

FIG. 2: Flow cytometric plots showing surface expression of CD13 and SSEA4 on human somatic fibroblast cells after 13 days of culture with expression of Yamanaka factors and 4 weeks of further culture in the absence of expression of Yamanaka factors (“reversion” as defined herein). Plots labelled with “+SSEA4” show those cells which were identified as CD13− SSEA4+ at day 13. Plots labelled with “+ CD13” and “−” show those cells identified as CD13+ SSEA4− at day 13 and those not cultured with expression of Yamanaka factors, respectively (i.e. negative control cultures).

FIG. 3: Brightfield phase contrast images of human somatic fibroblast cells identified as CD13− SSEA4+ at day 13 of culture with expression of Yamanaka factors, after a further 16 days of culture in the absence of expression of Yamanaka factors (“reversion”).

FIG. 4: Bar graph showing the DNA methylation age (as determined using the Horvath epigenetic clock) of human somatic fibroblast cells after partial reprogramming and reversion according to the methods as defined herein. “+OSKM SSEA4” represents cells identified as SSEA4+ at day 13 of culture with expression of Yamanaka factors and further cultured in the absence of expression of Yamanaka factors according to the methods as defined herein. “+OSKM CD13” and “−OSKM CD13” represent cells identified as CD13+ at day 13 of culture and those not cultured with expression of Yamanaka factors, respectively (i.e. negative control cultures, error bars represent two standard deviations).

FIG. 5: Schematic of the transient reprogramming experiment.

FIG. 6: Morphology of cells during and after transient reprogramming. After the doxycycline treatment, cells became iPSC-like and were forming colony structures. Cells returned to fibroblast-like morphology after being grown in the absence of doxycycline.

FIG. 7: Principal component analysis of the methylomes of transiently reprogrammed cells, fibroblasts, reprogramming cells and iPSCs. PC1 separates cells based on extent of reprogramming and suggests that transiently reprogrammed cells resemble fibroblasts.

FIG. 8: DNA methylation levels across the Oct4 locus. Grey rectangles denote promoter elements (from the Ensembl regulatory build) near the Oct4 gene (black rectangle). The Oct4 promoter is demethylated in iPSCs, however, it remained hypermethylated in transiently reprogrammed cells.

FIG. 9: DNA methylation levels across the FSP1 locus. Grey rectangles denote promoter elements (from the Ensembl regulatory build) near the FSP1 gene (black rectangle). The FSP1 promoter is hypermethylated in iPSCs, however, it remained demethylated in transiently reprogrammed cells.

FIG. 10: Principal component analysis of the transcriptomes of transiently reprogrammed cells, fibroblasts, reprogramming cells and iPSCs. PC1 separates cells based on extent of reprogramming and suggests that transiently reprogrammed cells resemble fibroblasts.

FIG. 11: Mean fibroblast specific protein 1 (FSP1) expression levels. FSP1 is highly expressed in transiently reprogrammed cells, control groups and reference fibroblasts, and is lowly expressed in iPSCs. Error bars represent the standard deviation.

FIG. 12: Mean Nanog expression levels. Nanog is not expressed in transiently reprogrammed cells, control groups and reference fibroblasts, and is expressed in iPSCs. Error bars represent the standard deviation.

FIG. 13: Mean DNA methylation age of samples. Error bars represent standard deviation. Transient reprogramming rejuvenated transcription age by up to 30-40 years relative to the control groups. Maximum rejuvenation was observed with 13 days of doxycycline treatment.

FIG. 14: Boxplots of H3K9me3 levels in individual cells measured by immunofluorescence. H3K9me3 levels decrease with age and were restored by transient reprogramming.

FIG. 15: Mean transcription age of samples. Error bars represent standard deviation. Transient reprogramming rejuvenated transcription age by approximately 30-40 years relative to the control groups. Rejuvenation was observed for all lengths of doxycycline treatment.

FIG. 16: Mean expression of collagen genes. Error bars represent standard deviation. P-values were calculated with DESeq2. * p<0.05, *** p<0.001. Transient reprogramming increases expression of some collagen genes.

FIG. 17: Boxplots of type I collagen levels in individual cells measured by immunofluorescence. Collagen levels decrease with age and were restored by 10 days of transient reprogramming.

### DETAILED DESCRIPTION OF THE INVENTION

According to a first aspect of the invention, there is provided a method of reprogramming a somatic cell to a pluripotent-like or rejuvenated state, comprising:
i) culturing said somatic cell in the presence of one or more Yamanaka factors for a period of at least 5 days, and/or until expression of a pluripotency marker is detectable on the surface of or within said somatic cell, and/or until a somatic cell lineage-specific marker is no longer detectable on the surface of said somatic cell;
ii) further culturing said somatic cell in the absence of said one or more Yamanaka factors until expression of said pluripotency marker has reduced on the surface of or within said somatic cell, and/or until expression of a somatic cell lineage-specific marker is detected on the surface of said somatic cell.

As will be appreciated from the present disclosures, contrasting to that previously known, it has been surprisingly shown herein that reprogramming of a somatic cell can be performed when said somatic cell is cultured in the presence of Yamanaka factors for a prolonged period of time. For example, Sarkar et al. (2019), bioRxiv 573386 (doi: https://doi.org/10.1101/_573386) have previously shown that transient reprogramming of somatic cells using a cocktail of OCT4, KLF4, c-MYC, SOX2, LIN28 and NANOG-encoding mRNA can be achieved with cultures of up to 4 days. As such, it has been proposed that day 5 of culture in the presence of these factors represents the “point of no return” for somatic cell reprogramming. After this “point of no return” at 5 days of culture in the presence of Yamanaka factors, it is suggested that the epigenetic signature which defines cell lineage identity is erased and reprogramming to an induced pluripotent stem (iPS) cell-like state is irreversible. Thus, according to Sarkar et al., in order to partially reprogram a somatic cell to a pluripotent-like or rejuvenated state or to a more pluripotent state, culturing of said somatic cell in the presence of Yamanaka factors must be done transiently (i.e. less than 5 days) and only during the “initiation” phase of iPS cell reprogramming.

During the process of iPS cell reprogramming, somatic cells are converted or de-differentiated into pluripotent stem cells. Such iPS cells are similar to natural pluripotent stem cells (e.g. embryonic stem (ES) cells) in many respects, including in their ability to differentiate into multiple cell types. However, during iPS cell reprogramming, DNA methylation age is reset to zero years old regardless of the age of the donor tissue from which the somatic cell was obtained. As such, the process of iPS cell reprogramming resets the epigenetic signature of the somatic cell to an embryonic-like state and causes loss of somatic cell lineage identity.

Thus, according to certain embodiments of the present invention, there are provided herein methods for reprogramming a somatic cell to a pluripotent-like or rejuvenated state (in particular a rejuvenated state), wherein said reprogramming is incomplete reprogramming and/or is partial reprogramming and/or is transient reprogramming. It will be appreciated that reference herein to “incomplete” and/or “partial” and/or “transient” reprogramming is compared to a cell with a high level of potency (e.g. an ES cell or an iPS cell). In a further embodiment, said reprogramming of a somatic cell is incomplete and/or partial and/or transient reprogramming compared to an iPS cell.

References herein to “somatic cell” refer to any type of cell that makes up the body of an organism, excluding germ cells and undifferentiated stem cells. Somatic cells may therefore include, for example, skin, heart, muscle, nerve, bone or blood cells. In one embodiment of the present invention, the somatic cell is a skin cell. In a further embodiment, the somatic cell is a cell from connective tissue, such as a fibroblast cell. In a yet further embodiment, the somatic cell is a blood cell. In one embodiment, the somatic cell is a bone marrow cell. Thus, it will be appreciated that in certain embodiments, the somatic cell may form blood or a part of blood. In another embodiment, the somatic cell is a nerve cell, such as a cell from the central and/or peripheral nervous system. Thus, in one embodiment, the cell is a neurone. In a further embodiment, the cell is a sensory neurone. In an alternative embodiment, the cell is a motor neurone. In another embodiment, the cell is an interneuron. In a further embodiment, the neurone is a brain cell. In a yet further embodiment, the cell is a pancreatic cell. Thus, in one embodiment, the cell is a pancreatic alpha cell. In an alternative embodiment, the cell is a pancreatic beta cell. In another embodiment, the cell is a pancreatic delta cell. In a further embodiment, the cell is a pancreatic F cell. In a yet further embodiment, the cell is a heart cell. Thus, in one embodiment, the cell is a cardiac myocyte (also known as a cardiac muscle cell, cardiomyocyte and myocardiocyte). In another embodiment, the cell is a sinatrial, or pacemaker, cell.

In one embodiment, the somatic cell is from an animal. In a further embodiment, the somatic cell is from a mammal. In a further embodiment, the mammal is a human. Thus, in a particular embodiment, the somatic cell is from a human and is a human somatic cell. In an alternative embodiment, the mammal is a mouse and the somatic cell is a mouse somatic cell. In a further alternative embodiment, the somatic cell is from a non-human mammal, such as a cat, dog or horse. For example, the rejuvenating properties of the somatic cells of the invention find particular utility in prolonging the life of a pet.

In a further embodiment, the incomplete and/or partial and/or transient reprogramming comprises the somatic cell in the presence of one or more Yamanaka factors for a period of time considered to be within the initiation and/or maturation phase of iPS cell reprogramming. In a yet further embodiment, the incomplete and/or partial and/or transient reprogramming comprises culturing the somatic cell in the presence of one or more Yamanaka factors at a time point considered to be prior to the stabilisation phase of iPS cell reprogramming. In a particular embodiment, the incomplete and/or partial and/or transient reprogramming comprises culturing the somatic cell in the presence of one or more Yamanaka factors at a time point considered to be in the maturation phase of reprogramming. Thus, in certain embodiments, the culturing in the presence of one or more Yamanaka factors is not performed in the stabilisation phase of iPS cell reprogramming.

References herein to “incomplete/incompletely reprogramming” and/or “partial/partially reprogramming” and/or “transient/transiently reprogramming” refer to a process or processes whereby a somatic cell is reprogrammed to a pluripotent-like or rejuvenated state (in particular a rejuvenated state) which comprises a molecular signature or DNA methylation age of younger, or less, than the donor tissue or organism from which the somatic cell was obtained. A DNA methylation age of younger, or less, than the donor tissue or organism from which the somatic cell was obtained includes an epigenetic signature which corresponds to that of a somatic cell from an earlier point in the life cycle of the tissue or organism. Therefore, references herein to “incomplete” and/or “partial” reprogramming and/or “transient” reprogramming also refer to wherein the reprogrammed somatic cell comprises a molecular signature, such as an epigenetic signature, which corresponds to that of a somatic cell from an earlier point in the life cycle of the tissue or organism from which the somatic cell was obtained.

Thus, in one embodiment, the reprogrammed somatic cell comprises a molecular signature, such as an epigenetic signature, which corresponds to that of a somatic cell from an earlier point in the life cycle of the tissue and/or organism. In a further embodiment, the molecular signature, such as the epigenetic signature, corresponds to that of a somatic cell from an earlier time point in the life cycle of the tissue and/or organism from which it was obtained.

References herein to “incomplete”, “partial” or “transient” reprogramming further refer to wherein the somatic cell is reprogrammed to a pluripotent-like or rejuvenated state (in particular a rejuvenated state) which comprises a molecular signature of younger, or less aged, than the donor tissue or organism from which the somatic cell was obtained. A molecular signature of younger, or less aged, than the donor tissue or organism from which the somatic cell was obtained includes an epigenetic signature which corresponds to that of a somatic cell from an earlier time point in the life cycle of the tissue or organism. Further molecular signatures include: transcriptomic profiles, number of γ-H2AX foci, concentration of reactive oxygen species, enrichment of histone marks (e.g. H3K9me3 and H4K20me3), collagen protein levels, vimentin and E-cadherin protein levels, senescence-associated β-galactosidase activity, cell proliferation rate and/or karyotypic signatures.

In certain embodiments, the molecular signature, such as the epigenetic signature, of the reprogrammed, non-reprogrammed somatic cell and/or reference cell (e.g. an iPS cell) is determined using the Horvath epigenetic clock. In further embodiments, the DNA methylation age of the reprogrammed somatic cell, non-reprogrammed somatic cell and/or reference cell is determined using the Horvath epigenetic clock. The Horvath epigenetic clock can be used as an age estimation method based on DNA methylation at CpG dinucleotide motifs in the DNA. DNA methylation age (further known as a “predicted age”) is characterised by the following properties: it is close to zero for ES and iPS cells; it correlates with cell passage number; it gives rise to a highly heritable measure of age acceleration; and it is applicable to chimpanzee tissues. The DNA methylation age of blood has been shown to predict all-cause mortality in later life, even after adjusting for known risk factors, suggesting that it is related to processes that cause ageing. Similarly, markers of physical and mental fitness have been associated with the epigenetic clock. One particular feature of the Horvath epigenetic clock is its high accuracy and applicability to a broad spectrum of tissues and cell types. Since it allows one to contrast the ages of different tissues and cells from the same subject (including a reprogrammed somatic cell with a non-reprogrammed somatic cell from the same tissue or a pluripotent cell, such as an iPS cell), it can be used to identify tissues and cells that show evidence of accelerated age due to disease. Furthermore, the Horvath epigenetic clock may be used to identify any change in DNA methylation age caused by treatment, such as reprogramming.

In further embodiments, the molecular signature as defined herein is determined using the transcriptome clock. Thus, in one embodiment, the molecular signature is determined using gene expression signatures or a gene expression signature. In a further embodiment, the transcriptome clock is determined using the method as described in Fleischer et al. (2018) Genome Biology 19, 221.

In one embodiment, the molecular signature and/or DNA methylation age of the reprogrammed somatic cell is younger, or less, than that of a somatic cell or the somatic cell prior to reprogramming from the same tissue or organism from which the somatic cell was obtained. In a further embodiment, the molecular signature and/or DNA methylation age of the reprogrammed somatic cell is in the form of an epigenetic signature indicative of a younger, or less aged, somatic cell or non-reprogrammed somatic cell from the same tissue or organism from which the somatic cell was obtained. In certain embodiments, the DNA methylation age and/or molecular signature, such as epigenetic signature, of the reprogrammed somatic cell is compared to that of a somatic cell from another tissue or organism (a “reference”). In such instances it will be appreciated that the DNA methylation age and/or molecular signature, such as epigenetic signature, of the reprogrammed somatic cell may be compared to a reference cell, tissue or organism which is the same age, older or younger than the tissue or organism from which the somatic cell was obtained. In an alternative embodiment, the DNA methylation age and/or molecular signature, such as epigenetic signature, of the reprogrammed somatic cell is compared to a pluripotent cell, such as an iPS cell.

In further embodiments, the DNA methylation age as calculated using the Horvath epigenetic clock and/or the molecular signature, such as the epigenetic signature, of the reprogrammed somatic cell indicates an age or DNA methylation age of at least 10 years, at least 15 years, at least 20 years, at least 25 years, at least 30 years, at least 35 years or at least 40 years younger, or less, than the non-reprogrammed somatic cell. In further embodiments, the molecular signature, such as the epigenetic signature, and/or DNA methylation age of the reprogrammed somatic cell indicates an age at least 10 years, at least 15 years, at least 20 years, at least 25 years, at least 30 years, at least 35 years or at least 40 years younger, or less, than a somatic cell from the tissue or organism from which the reprogrammed somatic cell was obtained. In a further embodiment, the molecular signature, such as the epigenetic signature, or DNA methylation age indicates an age of at least 20 years younger, or 20 years less, than a non-reprogrammed somatic cell, or a somatic cell from the same tissue or organism from which the reprogrammed somatic cell was obtained. In a further embodiment, the molecular signature, such as the epigenetic signature, or DNA methylation age indicates an age of at least 30 years younger, or 30 years less, than a non-reprogrammed somatic cell, or a somatic cell from the same tissue or organism from which the reprogrammed somatic cell was obtained. In a further embodiment, the molecular signature, such as the epigenetic signature, or DNA methylation age indicates an age of at least 40 years younger, or 40 years less, than a non-reprogrammed somatic cell, or a somatic cell from the same tissue or organism from which the reprogrammed somatic cell was obtained.

In further embodiments, the DNA methylation age as calculated using the Horvath epigenetic clock and/or the molecular signature, such as the epigenetic signature, of the reprogrammed somatic cell indicates an age or DNA methylation age of at least 10%, at least 20%, at least 30%, at least 40%, at least 50%, at least 60% younger, at least 70% younger, at least 80% younger or at least 90% younger, or less, than the non-reprogrammed somatic cell. In further embodiments, the molecular signature, such as the epigenetic signature, or DNA methylation age of the reprogrammed somatic cell indicates an age at least 10%, at least 20%, at least 30%, at least 40%, at least 50%, at least 60% younger, at least 70% younger, at least 80% younger or at least 90% younger, or less, than a somatic cell from the tissue or organism from which the reprogrammed somatic cell was obtained. In a further embodiment, the molecular signature, such as the epigenetic signature, or DNA methylation age indicates an age of at least 10% younger, or 10% less, than a non-reprogrammed somatic cell, or a somatic cell from the same tissue or organism from which the reprogrammed somatic cell was obtained. In a further embodiment, the molecular signature, such as the epigenetic signature, or DNA methylation age indicates an age of at least 40% younger, or 40% less, than a non-reprogrammed somatic cell, or a somatic cell from the same tissue or organism from which the reprogrammed somatic cell was obtained. In a yet further embodiment, the molecular signature, such as the epigenetic signature, or DNA methylation age indicates an age of at least 70% younger, or 70% less, than a non-reprogrammed somatic cell, or a somatic cell from the same tissue or organism from which the reprogrammed somatic cell was obtained.

It will be further appreciated that “incomplete/incompletely” and/or “partial/partially” and/or “transient/transiently” reprogramming as used herein include wherein the reprogrammed somatic cell retains and/or comprises the phenotype of a non-reprogrammed somatic cell. Such retention and/or comprising of the phenotype of a non-reprogrammed somatic cell includes wherein the expression of surface markers indicative of the cellular lineage or identity of the somatic cell are retained. Furthermore, such retention and/or comprising may also include wherein an epigenetic signature of the non-reprogrammed somatic cell lineage or identity is retained and/or comprised by the reprogrammed somatic cell.

Therefore, in one embodiment, the reprogrammed somatic cell retains the phenotype of the non-reprogrammed somatic cell. In a further embodiment, the reprogrammed somatic cell comprises the phenotype of the non-reprogrammed somatic cell. In a yet further embodiment, the reprogrammed somatic cell retains and/or comprises the phenotype of a non-reprogrammed somatic cell of the tissue from which the reprogrammed somatic cell was obtained. In another embodiment, the reprogrammed somatic cell retains and/or comprises a phenotype and/or epigenetic signature indicative of the cellular lineage or identity of the somatic cell.

References herein to one or more Yamanaka factors include one or more of: OCT4, KLF4, c-MYC and SOX2. In one embodiment, said one or more Yamanaka factors may additionally comprise LIN28 and NANOG. In an alternative embodiment, the one or more Yamanaka factors may be selected from one or more, two or more, three or more, four or more, five or more, six or more, seven or more, eight or more, or all of: OCT4, KLF4, c-MYC, SOX2, LIN28, NANOG, ESSRRB, NR5A2 and/or C/EBPα. In a further embodiment, the one or more Yamanaka factors are selected from: OCT4, KLF4, c-MYC and/or SOX2. In a yet further embodiment, the one or more Yamanaka factors are selected from: OCT4, KLF4 and/or SOX2. In an alternative embodiment, the one or more Yamanaka factors are selected from: OCT4, SOX2 and/or ESRRB. In an alternative embodiment, the one or more Yamanaka factors are selected from: KLF4, SOX2 and/or NR5A2. In an alternative embodiment, the one or more Yamanaka factors are selected from: OCT4, SOX2, KLF4, c-MYC and/or C/EBPα. In an alternative embodiment, the one or more Yamanaka factors are selected from: OCT4, KLF4 and/or c-MYC. In a further embodiment, the one or more Yamanaka factors are selected from: OCT4 and/or KLF4. In an alternative embodiment, the one or more Yamanaka factors are selected from: OCT4, SOX2, LIN28 and/or NR5A2. In a further embodiment, the one or more Yamanaka factors are selected from: OCT4 and/or SOX2. In an alternative embodiment, the one or more Yamanaka factors is selected from: OCT4, SOX2 and/or NR5A2. In a further embodiment, the one or more Yamanaka factors is: OCT4.

In a further embodiment, the method of reprogramming a somatic cell as defined herein comprises culturing said somatic cell in the presence of one or more Yamanaka factors for a period of at least 5 days. In a yet further embodiment, the somatic cell is cultured in the presence of one or more Yamanaka factors for a period of at least 6 days, at least 7 days, at least 8 days, at least 9 days, at least 10 days, at least 11 days, at least 12 days, at least 13 days, at least 14 days, at least 15 days or at least 16 days. In a particular embodiment, the somatic cell is cultured in the presence of one or more Yamanaka factors for at least 13 days. In a yet further embodiment, the somatic cell is cultured in the presence of one or more Yamanaka factors for a period of no more than 17 days, no more than 16 days, no more than 15 days or no more than 14 days. In one particular embodiment, the somatic cell is cultured in the presence of one or more Yamanaka factors for 13 days. In an alternative embodiment, the somatic cell is cultured in the presence of one or more Yamanaka factors for 15 days. In a further alternative embodiment, the somatic is cultured in the presence of one or more Yamanaka factors for 17 days.

References herein to culture of the somatic cell in the presence of one or more Yamanaka factors for 17 days will be appreciated to relate to a period of time which should not be exceeded when following exactly the protocols of the method as described herein. It will be further appreciated that the period for which the somatic cell is cultured in the presence of said one or more Yamanaka factors can vary depending on the identity of said somatic cell. For example, if the somatic cell is a fibroblast cell, culturing in the presence of one or more Yamanaka factors may be for at least 5 days, at least 13 days, at least 15 days, no more than 17 days, no more than 15 days or for 13, 15 or 17 days. Alternatively, if the cell is not a fibroblast cell, culturing in the presence of one or more Yamanaka factors may be for fewer days than those defined herein, or for more days than those defined herein.

In one embodiment, the method of reprogramming a somatic cell as defined herein comprises culturing said somatic cell in the presence of one or more Yamanaka factors until expression of a pluripotency marker is detectable on the surface of or within the somatic cell. It will be appreciated that references herein to “a pluripotency marker” may include any marker expressed by the somatic cell undergoing reprogramming which is associated with pluripotency or which is associated with a pluripotent-like or rejuvenated state (in particular a rejuvenated state). Such markers may be expressed on the surface of the somatic cell or may be expressed intracellularly (i.e. “in”, e.g. as in the case of pluripotency-associated transcription factors). In one embodiment, the pluripotency marker is selected from: OCT4, SOX2, NANOG, KLF4, TRA-1-60, TRA-1-81, TRA-1-54, SSEA1 and/or SSEA4. In a further embodiment, the pluripotency marker is a transcription factor and expression is detected intracellularly, and the pluripotency marker is selected from: OCT4, SOX2, NANOG and/or KLF4. In a yet further embodiment, the pluripotency marker is detected on the surface of the somatic cell and is selected from TRA-1-60, TRA-1-81, TRA-2-54, SSEA1, SSEA3 and/or SSEA4.

In a particular embodiment, the pluripotency marker detected on the surface of the somatic cell is SSEA4 (stage-specific embryonic antigen-4).

Stage-specific embryonic antigen-4 (SSEA4) is a glycolipid carbohydrate antigen expressed on the surface of human embryonal carcinoma (EC), embryonic germ (EG), undifferentiated ES and iPS cells and a subset of mesenchymal stem cells, as well as rhesus monkey ES cell lines. Expression of SSEA4 is downregulated following differentiation of human EC, ES and iPS cells. As such, SSEA4 surface expression may be used as a marker of de-differentiation or reprogramming of a somatic cell to a pluripotent-like or rejuvenated state (in particular a rejuvenated state).

In an alternative embodiment, the pluripotency marker detected on the surface of the somatic cell is SSEA1 (stage-specific embryonic antigen-1, also known as CD15).

Stage-specific embryonic antigen-1 (SSEA1) is a lactoseries oligosaccharide expressed on the surface of mouse embryonic carcinoma, embryonic stem, and germ cells, but only expressed on human germ cells. Expression of SSEA1 on human cells increases upon differentiation, while differentiation of mouse cells leads to decreased expression.

In an alternative embodiment, the pluripotency marker detected on the surface of the somatic cell is SSEA3 (stage-specific embryonic antigen-3).

Stage-specific embryonic antigen-3 (SSEA3) is a glycosphingolipid oligosaccharide composed of five carbohydrate units connected to a sphingolipid. Such sphingolipids function as key players in cell signalling and SSEA3 has been shown to play a key role in identifying many types of mammalian cells with pluripotent and stem cell-like characteristics.

In an alternative embodiment, the pluripotency marker detected on the surface of the somatic cell is selected from: TRA-1-60, TRA-1-81 and/or TRA-2-54. TRA-1-60, TRA-1-81 and TRA-2-54 are keratin sulphate antigens expressed on the surface of human ES cells.

In further embodiments, the pluripotency marker is a transcription factor, such as a transcription factor associated with pluripotency or a pluripotent-like or rejuvenated state (in particular a rejuvenated state). Thus, in one embodiment, the pluripotency marker is OCT4.

Octamer-binding transcription factor 4 (OCT4) is a homeodomain transcription factor of the POU family encoded by the POU5F1 gene in humans. It is critically involved in the self-renewal of undifferentiated embryonic stem cells and is initially active as a maternal factor in the oocyte and remains active in embryos throughout the preimplantation period. Gene knockdown of OCT4 promotes differentiation, demonstrating a role for these factors in human embryonic stem cell self-renewal. Mouse embryos that are Oct4 deficient or have low expression levels of Oct4 fail to form the inner cell mass, lose pluripotency, and differentiate into trophectoderm. Therefore, the level of Oct4 expression in mice is vital for regulating pluripotency and early cell differentiation.

In a further embodiment, the pluripotency marker is SOX2.

SRY (sex determining region Y)-box 2 (SOX2) is a transcription factor that is essential for maintaining self-renewal, or pluripotency, of undifferentiated embryonic stem cells. SOX2 is a member of the Sox family of transcription factors and has been shown to have a critical role in maintenance of embryonic and neural stem cells. SOX2 binds to DNA cooperatively with OCT4 at non-palindromic sequences to activate transcription of key pluripotency factors. Therefore, it will be appreciated that as described herein, OCT4 and SOX2 can be used interchangeably and/or cooperatively.

In a yet further embodiment, the pluripotency marker is NANOG.

NANOG is a homeobox protein which is a transcription factor that helps ES cells maintain pluripotency by suppressing cell determination factors. NANOG is thought to function in concert with other factors such as OCT4 and SOX2 to establish ES cell identity.

In one embodiment, the pluripotency marker is KLF4.

Kruppel-like factor 4 (KLF4, also known as gut-enriched Kruppel-like factor or GKLF) is a zinc-finger transcription factor involved in the regulation of proliferation, differentiation, apoptosis and somatic cell reprogramming. In ES cells, KLF4 has been demonstrated to be a good indicator of stem-like capacity and it has been suggested that the same is true in mesenchymal stem cells.

According to certain embodiments, it will be appreciated that when the pluripotency marker is a transcription factor (e.g. OCT4, SOX2, NANOG and/or KLF4), said pluripotency marker does not have the same identity as the one or more Yamanaka factors which the somatic cell is cultured in the presence of according to methods defined herein. It will be further appreciated that when the pluripotency marker is a transcription factor, expression of said pluripotency marker is not detected on the surface of the somatic cell and expression of said transcription factor pluripotency marker in the somatic cell may be detected by expression and/or activation of a reporter or downstream effector of said transcription factor.

In a further embodiment, the method of reprogramming a somatic cell as defined herein comprises culturing said somatic cell in the presence of one or more Yamanaka factors until expression of a somatic cell lineage-specific marker (e.g. CD13) is no longer detected on the surface of the somatic cell. In an alternative embodiment, the culturing of the somatic cell in the presence of one or more Yamanaka factors is until expression of a somatic cell lineage-specific marker is downregulated or reduced on the surface of the somatic cell. It will be appreciated that references herein to “no longer detected”, “downregulated” and “reduced” encompass any change in the surface expression, including loss, of the marker compared to a non-reprogrammed somatic cell or compared to the somatic cell prior to reprogramming, wherein the non-reprogrammed somatic cell comprises higher, or more, expression of the marker. It will be further appreciated that such references herein may also be compared to a reference pluripotent cell, such as an ES or iPS cell.

References herein to “culturing in the presence of one or more Yamanaka factors” will be appreciated to include providing said one or more Yamanaka factors as defined herein to the somatic cell in culture in any form. Such culturing in the presence of one or more Yamanaka factors may, in one embodiment, comprise addition of one or more Yamanaka factors in protein or peptide form to the culture medium or media. In a further embodiment, culturing in the presence of one or more Yamanaka factors comprises culturing the somatic cell in the presence of cells expressing the one or more Yamanaka factors as defined herein. In further embodiments, the culturing in the presence of one or more Yamanaka factors comprises expression of the one or more Yamanaka factors in the somatic cell. Thus, according to one embodiment, culturing in the presence of one or more Yamanaka factors as defined herein comprises expression from the endogenous one or more Yamanaka factor-encoding genes of the somatic cell. According to this embodiment, the expression of one or more Yamanaka factors in the somatic cell does not comprise transfection, transduction or introduction of exogenous sequences. In a further embodiment, expression of one or more Yamanaka factors in the somatic cell comprises stimulated expression using a compound and/or treatment which upregulates or “turns on” expression of one or more Yamanaka factor-encoding genes. Thus, in one embodiment, culturing in the presence of one or more Yamanaka factors comprises addition of a compound known to cause expression of one or more Yamanaka factor-encoding genes. In a particular embodiment, the compound is known to cause expression of the one or more Yamanaka factor-encoding genes in the somatic cell.

In an alternative embodiment, culturing in the presence of one or more Yamanaka factors comprises introducing into the somatic cell exogenous sequences encoding the one or more Yamanaka factors as defined herein. Thus, in one embodiment, culturing in the presence of one or more Yamanaka factors comprises expression of the one or more Yamanaka factors from an exogenous sequence or from exogenous sequences.

In one embodiment, the exogenous sequences encoding the one or more Yamanaka factors as defined herein are in the form of Yamanaka factor-encoding mRNA. Thus, in one embodiment, the culturing of the somatic cell in the presence of one or more Yamanaka factors comprises culturing the somatic cell in the presence of Yamanaka factor-encoding mRNA. In a further embodiment, the culturing of the somatic cell in the presence of Yamanaka factors comprises providing the somatic cell with Yamanaka factor-encoding mRNA.

In one embodiment, the exogenous sequences encoding the one or more Yamanaka factors as defined herein are introduced into the somatic cell by transfection. In an alternative embodiment, the exogenous sequences are introduced into the somatic cell by transduction, such as viral transduction. It will be appreciated that viral transduction is not limited to any specific virus, however, in one particular non-limiting embodiment, the viral transduction is lentiviral transduction. In an alternative embodiment, the viral transduction is retroviral transduction. In one embodiment, the exogenous one or more Yamanaka factor-encoding sequences as defined herein may be introduced into the somatic cell in the form of a vector transfected into the somatic cell. In one embodiment, the vector is a transposon vector. Vectors suitable for introduction of expression of one or more Yamanaka factors as used herein into a host cell, such as a somatic cell, are well known in the art. A vector may also contain various regulatory/responsive sequences or elements that control the transcription and/or translation of the target sequence (such as those responsive elements which allow for inducible expression as defined herein). Examples of vectors include: viral vectors, transposon vectors, plasmid vectors or cosmid vectors. It will also be appreciated that said Yamanaka factors may be introduced into a host cell, such as the somatic cell, by CRISPR/Cas-9 methodology. Such methodology may be drug- (i.e. doxycycline (dox)) inducible or non-inducible CRISPR/Cas-9 methodology and is well known to the skilled person.

Transposon vectors utilise mobile genetic elements known as transposons to move target sequences to and from vectors and chromosomes using a “cut and paste” mechanism. Examples of transposon vectors include PiggyBac vectors (System Biosciences) or EZ-Tn5™ Transposon Construction vectors (Illumina, Inc.).

Viral vectors consist of DNA or RNA inside a genetically-engineered virus. Viral vectors may be used to integrate the target sequence into the host cell genome (i.e. integrating viral vectors). Examples of viral vectors include adenoviral vectors, adenoviral-associated vectors, retroviral vectors or lentiviral vectors (e.g. HIV). Viral vectors may be introduced into the host cell, such as a somatic cell, by way of viral transduction. Thus, according to one embodiment, expression of the one or more Yamanaka factors in the somatic cell and/or culturing in the presence of one or more Yamanaka factors comprises integration of the one or more Yamanaka factor-encoding sequences into the genome of the somatic cell. In a further embodiment, expression of the one or more Yamanaka factors in the somatic cell and/or culturing in the presence of one or more Yamanaka factors comprises use of a viral vector to integrate the one or more Yamanaka factor-encoding sequences into the somatic cell genome.

Plasmid vectors consist of generally circular, double-stranded DNA. Plasmid vectors, like most engineered vectors, have a multiple cloning site (MCS), which is a short region containing several commonly used restriction sites which allows DNA fragments of interest to be easily inserted.

References herein to “transfection” refer to a process by which the vector is introduced into the host cell (e.g. the somatic cell) so that the target sequence can be expressed. Methods of transfecting the host cell with the vector include electroporation, sonoporation or optical transfection, which are well known in the art.

In one embodiment, the expression of the one or more Yamanaka factors as defined herein may be introduced and/or provided to the somatic cell in the form of an expression cassette. In a further embodiment, culturing in the presence of one or more Yamanaka factors comprises introduction of one or more Yamanaka factor-encoding sequences into the somatic cell in the form of an expression cassette. Thus, in one embodiment, expression of the one or more Yamanaka factors as defined herein is from an expression cassette. Such an expression cassette may comprise, in a particular embodiment, mRNA-derived sequences encoding the one or more Yamanaka factors as described herein. In a further embodiment, the expression cassette additionally comprises a sequence encoding a protein or marker which allows for the identification of expression of the expression cassette. In a particular embodiment, said protein or marker allowing for the identification of expression is a fluorescent protein. In a certain embodiment, the fluorescent protein is green fluorescent protein (GFP).

Thus, in one embodiment, the somatic cell may be selected based on expression of a protein or marker comprised in the expression cassette. In a particular embodiment, the somatic cell is selected based on expression of a fluorescent protein (e.g. GFP) which allows for the identification of expression. It will be appreciated that “selected” as used herein may include flow cytometric methods, such as fluorescence-activated cell sorting (FACS).

In a further embodiment, the marker which allows for the identification of expression may be selected from a drug resistance gene. Examples of drug resistance genes may include: a puromycin resistance gene, an ampicillin resistance gene, a neomycin resistance gene, a tetracycline resistance gene, a kanamycin resistance gene or a chloramphenicol resistance gene. Cells can be cultured in a medium containing the appropriate drug (i.e. a selection medium) and only those cells which incorporate and express the drug resistance gene will survive. Therefore, by culturing cells using a selection medium, it is possible to easily select cells comprising a drug resistance gene.

Alternative markers which allow for the identification of expression include chromogenic enzyme genes. Examples of chromogenic enzyme genes include: β-galactosidase gene, β-glucuronidase gene, alkaline phosphatase gene, or secreted alkaline phosphatase SEAP gene. Cells expressing these chromogenic enzyme genes can be detected by applying the appropriate chromogenic substrate (e.g. X-gal for β galatosidase) so that cells expressing the marker gene will produce a detectable colour (e.g. blue in a blue-white screen test).

In a further embodiment, the expression cassette is an inducible expression cassette which allows for the expression or co-expression of the one or more Yamanaka factor-encoding sequences upon induction of expression with a suitable compound or treatment. Such inducible expression cassettes will be appreciated to include a responsive element which allows for expression of the cassette either by promoting transcription and/or translation or removing inhibition from transcription and/or translation. In one embodiment, said responsive element is a tetracycline responsive element. Thus, in certain embodiments, the inducible expression cassette allows for the expression or co-expression of one or more Yamanaka factor-encoding sequences upon addition of an antibiotic, such as a tetracycline, in particular a doxycycline (as exemplified in the data presented herein).

It will therefore be appreciated, that according to one embodiment, the culturing of the somatic cell in the presence of one or more Yamanaka factors comprises addition of a compound or treatment capable of inducing expression from the inducible expression cassette. In certain embodiments, the culturing of the somatic cell in the presence of one or more Yamanaka factors comprises the addition of tetracycline.

In one embodiment, the exogenous sequences encoding the one or more Yamanaka factors as defined herein are in the form of proteins expressed from Yamanaka factor-encoding mRNA. It will be appreciated that the expressed proteins (i.e. proteins expressed from Yamanaka factor-encoding mRNA) may be directly transferred (i.e. transfected) into the somatic cell using suitable protein delivery methodology. It will be appreciated that such suitable methodology for direct transfer of proteins into cells is well known to the skilled person and includes functional twin-arginine translocation (Tat) systems. Alternatively, said proteins may be directly transferred to the somatic cell by targeted delivery systems, such as nanoparticle delivery systems. Once again, such targeted delivery systems are well known to the skilled person.

In a yet further embodiment, the method of reprogramming a somatic cell as defined herein comprises further culturing said somatic cell in the absence of said one or more Yamanaka factors for a period of at least 2 weeks. In a further embodiment, the somatic cell is further cultured in the absence of said one or more Yamanaka factors for a period of at least 2.5 weeks, at least 3 weeks, at least 3.5 weeks or at least 4 weeks. In yet further embodiments, the somatic cell is further cultured in the absence of said one or more Yamanaka factors for no more than 5 weeks, no more than 4 weeks, no more than 3 weeks or no more than 2.5 weeks. In a particular embodiment, the somatic cell is further cultured in the absence of said one or more Yamanaka factors for 4 weeks. In an alternative embodiment, the somatic cell is further cultured in the absence of said one or more Yamanaka factors for 3 weeks. In another alternative embodiment, the somatic cell is further cultured in the absence of said one or more Yamanaka factors for 2 weeks.

In one embodiment, the method of reprogramming a somatic cell as defined herein comprises further culturing said somatic cell in the absence of said one or more Yamanaka factors until expression of a pluripotency marker is downregulated or has reduced on the surface of or within the somatic cell. In a further embodiment, the somatic cell is further cultured in the absence of said one or more Yamanaka factors until expression of a pluripotency marker is no longer detectable on the surface of or within the somatic cell. Such references according to this embodiment to “downregulated”, “reduced” or “no longer detectable” will be appreciated to be relative to the somatic cell prior to further culturing in the absence of said one or more Yamanaka factors and/or to a reference pluripotent cell.

It will be appreciated that the pluripotency marker according to these embodiments can be the same or different to the pluripotency marker detected on the surface of or within the somatic cell cultured in the presence of one or more Yamanaka factors. Thus, in one particular embodiment, the expression of a pluripotency marker which is downregulated or no longer detected on the surface or within the somatic cell upon culture in the absence of one or more Yamanaka factors is said pluripotency marker which is detectable on the surface of or within the somatic cell following culture in the presence of one or more Yamanaka factors.

In a further embodiment, the method of reprogramming a somatic cell as defined herein comprises further culturing said somatic cell in the absence of said one or more Yamanaka factors until expression of a somatic cell lineage-specific marker (e.g. CD13) is detectable on the surface of the somatic cell. In an alternative embodiment, the further culturing in the absence of said one or more Yamanaka factors is until expression of a somatic cell lineage-specific marker is upregulated or increased on the surface of the somatic cell. References herein to “upregulated” and “increased” encompass any change, including gain, in the surface expression of the marker compared to the somatic cell prior to the step of further culturing in the absence of said one or more Yamanaka factors. In such instances, it will be appreciated that the somatic cell prior to the step of further culturing in the absence of said one or more Yamanaka factors comprises lower, less or no expression of the somatic cell lineage-specific marker. Further references herein to “detectable”, “upregulated” and “increased” may also be compared to a reference pluripotent cell, such as an iPS cell. In an alternative embodiment, the further culturing in the absence of said one or more Yamanaka factors is until expression of a somatic cell lineage-specific marker is restored compared to that of the reprogrammed somatic cell prior to the step of further culturing in the absence of said one or more Yamanaka factors, or compared to a somatic cell prior to culture in the presence of one or more Yamanaka factors, or compared to a non-reprogrammed somatic cell.

Thus, it will be appreciated that further culturing in the absence of expression of said one or more Yamanaka factors as defined herein can be referred to as “reversion” or “recovery”.

In a yet further embodiment, the further culturing of said somatic cell in the absence of said one or more Yamanaka factors comprises removing the compound or treatment capable of inducing expression from an inducible expression cassette. In certain embodiments, the further culturing of the somatic cell in the absence of said one or more Yamanaka factors comprises removing tetracycline. In an alternative embodiment, further culturing in the absence of said one or more Yamanaka factors comprises a compound or treatment capable of preventing or stopping expression from the inducible expression cassette.

According to another aspect of the invention, there is provided a reprogrammed somatic cell produced according to the methods as defined herein. It will be appreciated that references herein to “a” or “the” reprogrammed somatic cell include a single or small number of cells, as well as to a population of reprogrammed somatic cells, which may be large in number. Thus, it will be appreciated that any references herein to singular include plural and vice versa.

In one embodiment, the reprogrammed somatic cell produced according to the methods as defined herein comprises a DNA methylation age, epigenetic age or molecular signature that is younger, or less, than a non-reprogrammed somatic cell or a somatic cell from the tissue or organism from which the reprogrammed somatic cell has been obtained. In a further embodiment, the reprogrammed somatic cell comprises a molecular signature, such as an epigenetic signature, indicative of a younger, or lower, epigenetic age than a non-reprogrammed somatic cell or a somatic cell from the tissue or organism from which the reprogrammed somatic cell was obtained. In a particular embodiment, the reprogrammed somatic cell produced according to the methods as defined herein comprises a molecular signature, such as an epigenetic signature, similar to that of a somatic cell from an earlier point in the life-cycle of the tissue or organism from which the somatic cell was obtained. In further embodiments, the reprogrammed somatic cell produced according to the methods as defined herein comprises a phenotype and/or a molecular signature, such as an epigenetic signature, similar to that of a non-reprogrammed somatic cell.

### Compositions Comprising Reprogrammed Somatic Cells

According to a further aspect of the invention, there is provided a pharmaceutical composition comprising a reprogrammed somatic cell as defined herein.

According to a yet further aspect of the invention, there is provided a cosmetic composition comprising a reprogrammed somatic cell as defined herein.

According to certain embodiments, the pharmaceutical or cosmetic composition, in addition to comprising a reprogrammed somatic cell as defined herein, further comprises one or more pharmaceutically acceptable excipients. In further embodiments, the pharmaceutical or cosmetic composition, in addition to comprising a reprogrammed somatic cell produced according to the methods as defined herein, further comprises one or more pharmaceutically acceptable excipients.

Generally, the present pharmaceutical and cosmetic compositions will be utilised with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a composition comprising the reprogrammed somatic cell as defined herein in a discrete location, may be chosen from thickeners such as carboxymethylcellulose, polyvinylpyrrolidone, gelatine and alginates. Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16^{th} Edition).

The route of administration of pharmaceutical compositions as defined herein may be any of those commonly known to those of ordinary skill in the art. For example, the administration can be by any appropriate mode, including parenterally, intravenously, intramuscularly, intraperitoneally, dermally or transdermally. In particular embodiments, the pharmaceutical compositions as defined herein may be administered intravenously or transdermally.

Furthermore, the route of administration of cosmetic compositions as defined here may also be any of those commonly known to those of ordinary skill in the art. For example, administration can be by any appropriate mode, including those mentioned above. In particular embodiments, the cosmetic compositions as defined herein may be administered topically, dermally or transdermally.

### Therapeutic Uses and Methods

It will be appreciated from the disclosures presented herein that the methods and compositions of the present invention will find particular utility in the treatment and/or amelioration of age-related or degenerative diseases and/or disorders, or in the rejuvenation of a tissue or organ.

Thus, according to one aspect, there is provided a method comprising a reprogrammed somatic cell produced according to the methods as defined herein, for the treatment and/or amelioration of an age-related or degenerative disease or disorder, said method further comprising administering said reprogrammed somatic cell to a subject in need thereof. In another embodiment, there is provided a method comprising a reprogrammed somatic cell as defined herein for the treatment and/or amelioration of an age-related or degenerative disease or disorder, said method further comprising administering said reprogrammed somatic cell to a subject in need thereof. In one embodiment, the method comprising a reprogrammed somatic cell as defined herein is for the treatment and/or amelioration of an age-related or degenerative disease or disorder of the skin. In an alternative embodiment, the method comprising a reprogrammed somatic cell as defined herein is for the treatment or amelioration of an age-related or degenerative disease or disorder of the pancreas, such as for the treatment or amelioration of type 2 diabetes. In a further embodiment, the method comprising a reprogrammed somatic cell as defined herein is for the treatment and/or amelioration of an age-related disease or disorder, wherein the age-related disease or disorder is a neurodegenerative disorder. In a yet further embodiment, the method comprising a reprogrammed somatic cell as defined herein is for the treatment and/or amelioration of an age-related disease or disorder, wherein the age-related disease or disorder is a disease or disorder of the blood and/or bone marrow. In a still further embodiment, the method comprising a reprogrammed somatic cell as defined herein is for the treatment and/or amelioration of an age-related disease or disorder, wherein the age-related disease or disorder is of the heart. Thus, in one embodiment, the disease or disorder is cardiovascular disease. In a further embodiment, the disease or disorder is a cardiomyopathy. In another embodiment, the disease or disorder is ischaemic heart disease. In a yet further embodiment, the disease or disorder is cardiac arrhythmia. In another embodiment, the disease or disorder is heart failure.

In a further embodiment, there is provided the use of a reprogrammed somatic cell as defined herein and/or produced according to the methods as defined herein, in the treatment and/or amelioration of an age-related or degenerative disease or disorder. In a yet further embodiment, there is provided a method of producing a reprogrammed somatic cell as defined herein, for the treatment and/or amelioration of an age-related or degenerative disease or disorder.

According to a further aspect, there is provided a pharmaceutical composition as defined herein for use in the treatment and/or amelioration of a degenerative or age-related disease or disorder, or for use in the rejuvenation of a tissue or organ. In one embodiment, said pharmaceutical composition comprises a reprogrammed somatic cell as defined herein. In an alternative embodiment, said pharmaceutical composition comprises a reprogrammed somatic cell produced according to the methods as defined herein.

In one embodiment, the pharmaceutical composition for use comprising the reprogrammed somatic cell as defined herein or the reprogrammed somatic cell as defined herein, is for use in the treatment of the skin or for treatment and/or amelioration of a disease or disorder of the skin. Thus, in certain embodiments, the age-related disease or disorder comprises a disease or disorder of the skin. In a further embodiment, the treatment of the skin is to prevent, inhibit, reduce and/or reverse ageing of the skin. Examples of ageing of the skin include wrinkles, dryness, loss of elasticity, fragility and/or loss of barrier properties.

In an alternative embodiment, the pharmaceutical composition for use comprising the reprogrammed somatic cell as defined herein or the reprogrammed somatic cell as defined herein, is for use in the treatment and/or amelioration of a disease or disorder of the pancreas. Thus, in certain embodiments, the age-related disease or disorder comprises a disease or disorder of the pancreas. In a further embodiment, the disease or disorder of the pancreas is type 2 diabetes.

In a further embodiment, the pharmaceutical composition for use comprising the reprogrammed somatic cell as defined herein or the reprogrammed somatic cell as defined herein, is for use in the treatment and/or amelioration of a neurodegenerative disorder.

In a yet further embodiment, the pharmaceutical composition for use comprising the reprogrammed somatic cell as defined herein or the reprogrammed somatic cell as defined herein, is for use in the treatment and/or amelioration of a disease or disorder of the blood and/or bone marrow.

In a further embodiment, the pharmaceutical composition for use comprising the reprogrammed somatic cell as defined herein or the reprogrammed somatic cell as defined herein, is for use in the treatment and/or amelioration of a disease or disorder of the heart.

In further embodiments, the tissue or organ as defined herein is selected from: skin, liver, pancreas, heart, brain, central nervous system, peripheral nervous system, blood and/or bone marrow. Thus, according to one embodiment the tissue or organ is selected from blood and the treatment and/or amelioration comprises subjecting the blood or a blood cell to one or more of the methods defined herein and providing said blood or blood cell to a patient or subject in need thereof. In a further embodiment, the tissue or organ is selected from bone marrow and the rejuvenation comprises subjecting the bone marrow or a bone marrow cell to one or more of the methods defined herein and providing said bone marrow or bone marrow cell to a patient or subject in need thereof.

In a particular embodiment, the tissue or organ is selected from the liver and the methods and pharmaceutical composition as defined herein are for use in the rejuvenation of the liver. It will be appreciated that, according to this embodiment, such rejuvenation may comprise rejuvenating only part of the liver tissue or organ or a somatic cell from the liver tissue or organ and providing said rejuvenated liver tissue or liver tissue cell to a patient or subject in need thereof. In a further embodiment, the rejuvenated liver as defined herein may continue to rejuvenate or rejuvenate further in vivo.

In a further embodiment, the tissue or organ is selected from the heart and the methods and pharmaceutical composition as defined herein are for use in the rejuvenation of the heart or heart tissue. Thus, according to one embodiment, the tissue or organ is selected from the heart and the treatment and/or amelioration or rejuvenation comprises subjecting a heart cell, such as a cardiac myocyte, to one or more of the methods defined herein and providing said heart cell to a patient or subject in need thereof. In a further embodiment, the tissue or organ is selected from the heart and the rejuvenation comprises subjecting a heart cell, such as a cardiac myocyte, to one or more of the methods defined herein and providing said heart cell to a patient or subject in need thereof. In another embodiment, the tissue or organ is selected from the heart and the reprogrammed somatic cell as defined herein is a heart cell, such as a cardiac myocyte, and the treatment and/or amelioration or rejuvenation comprises providing said reprogrammed somatic heart cell to a patient or subject in need thereof.

It will be appreciated that, according to the embodiments described herein, the tissue or organ may be either from said patient or subject in need thereof, or alternatively derived from a donor subject.

It will be appreciated that references herein to a patient or subject in need thereof relate equally to animals and humans and that the invention finds particular utility in veterinary treatment of any of the above mentioned diseases, disorders and conditions which are also present in said animals.

It will be appreciated that references herein to “treatment” and “amelioration” include such terms as “prevention”, “reversal” and “suppression”. Furthermore, such references include administration of the reprogrammed somatic cell or composition comprising the reprogrammed somatic cell as defined herein prior to the onset of the disease or disorder. Administration of the reprogrammed somatic cell or composition comprising the reprogrammed somatic cell as defined herein may also be anticipated after the induction event of the disease or disorder, either before clinical presentation of said disease or disorder, or after symptoms manifest.

### Cosmetic Uses and Methods

According to one aspect of the invention, there is provided a cosmetic method of regenerating or rejuvenating skin comprising administration or application of a reprogrammed somatic cell as defined herein or a cosmetic composition as defined herein to a subject in need thereof.

In an alternative aspect, the cosmetic method is for rejuvenating a tissue or organ in need thereof, wherein the tissue or organ is not the skin. In another aspect, the cosmetic compositions as defined herein may be used for the rejuvenation of a tissue or organ in need thereof, wherein the tissue or organ is not the skin.

It will be appreciated that cosmetic compositions and methods comprising a reprogrammed somatic cell as defined herein may be suitably used for the regeneration or rejuvenation of the skin. Furthermore, such cosmetic compositions and methods may be useful for the reduction of scar formation or for the regeneration of connective tissue. Alternatively and/or additionally, cosmetic compositions as defined herein may be useful for the regeneration or rejuvenation of skin and/or connective tissue used in cosmetic surgery or after cosmetic surgery. Suitably, cosmetic compositions as defined herein are useful for the regeneration or rejuvenation of skin and/or connective tissue comprising reducing the age, such as the DNA methylation age or epigenetic age, or making younger, the skin and/or connective tissue.

Furthermore, cosmetic methods as defined herein may be useful for the regeneration of skin and/or connective tissue after cosmetic surgery. The cosmetic methods as defined herein are anticipated to find particular utility in the regeneration of skin and/or connective tissue used in cosmetic surgery.

It will be further appreciated that cosmetic compositions as defined herein may be administered and/or utilised prophylactically. For example, a cosmetic composition comprising a reprogrammed somatic cell as defined herein may be used at a point before a tissue and/or organism is considered aged and/or old, such as at an early time point in the life-cycle of the tissue and/or organism.

### Screening Methods

According to a further aspect, there is provided a method of screening for an age modulating agent, said method comprising:
(i) performing the method as defined herein in the presence and the absence of a test agent to generate a reprogrammed somatic cell; and
(ii) determining the molecular signature, such as the epigenetic signature, of the reprogrammed somatic cell,
   wherein a difference between the molecular signature determined for a reprogrammed somatic cell generated in the presence of the test agent and the molecular signature determined for a reprogrammed somatic cell generated in the absence of the test agent is indicative of the age modulating effect of said agent.

It will be appreciated that, according to this aspect of the invention, a test agent may comprise any compound, treatment, condition or process which may increase, speed-up or accelerate the ageing of a cell, tissue, organ or organism or, alternatively, may decrease, slow-down or decelerate the ageing of a cell, tissue, organ or organism. Thus, in one embodiment, the test agent accelerates, speeds-up or increases the ageing of a cell, tissue or organism. In an alternative embodiment, the test agent decelerates, slows-down or decreases the ageing of the cell, tissue or organism. In a further embodiment, the test agent decreases the effects of the reprogramming methods as defined herein. In another embodiment, the test agent increases the effects of the reprogramming methods as defined herein. In an alternative embodiment, the test agent prevents the reprogramming effects of the methods as defined herein.

In one embodiment, the difference between the molecular signature is determined between a reprogrammed somatic cell as defined herein or generated according to the methods as defined herein which has been exposed to the test agent and a reprogrammed somatic cell as defined herein or generated according to the methods as defined herein which has not been exposed to the test agent. Thus, in one embodiment, the difference between the molecular signature is determined for a somatic cell reprogrammed in the presence of the test agent and the molecular signature determined for a somatic cell reprogrammed in the absence of the test agent. In a further embodiment, the difference between the molecular signature is determined between a somatic cell reprogrammed according to the methods defined herein or a reprogrammed somatic cell as defined herein and a non-reprogrammed somatic cell exposed to the test agent. In a yet further embodiment, the difference between the molecular signature is determined between a somatic cell reprogrammed according to the methods defined herein or the reprogrammed somatic as defined herein exposed to the test agent and a non-reprogrammed somatic cell.

According to a yet further aspect, there is provided a method of screening for an age modulating factor or cellular process, said method comprising:
(i) reprogramming a somatic cell from a diseased tissue or organ according to the method as defined herein; and
(ii) determining the molecular signature, such as the epigenetic signature, of the reprogrammed somatic cell from a diseased tissue or organ and of a reprogrammed somatic cell as defined herein or of a non-reprogrammed somatic cell from said diseased tissue or organ,
   wherein a difference between the molecular signature determined for the reprogrammed somatic cell from a diseased tissue or organ and the molecular signature determined for the reprogrammed somatic cell as defined herein or the non-reprogrammed somatic cell from the diseased tissue or organ is indicative of the age modulating factor or cellular process associated with the disease.

In one embodiment, the age modulating factor is a factor expressed or present in the somatic cell which is involved in or modulates, or is suspected to be involved in or modulate, the age or ageing of the cell, tissue, organ or organism. In a further embodiment, the age modulating cellular process is a cellular process which is involved in or modulates, or is suspected to be involved in or modulate, the age or ageing of the cell, tissue, organ or organism. In a further embodiment, the age modulating factor or cellular process is involved in or modulates, or is suspected to be involved in or modulate, an age-related disease or disorder.

In one embodiment, the somatic cell is obtained from a diseased tissue or organ, wherein the disease is an age-related disease or disorder. In certain embodiments, the age-related disease or disorder is selected from an age-related disease or disorder as described herein.

Thus, in one embodiment, the difference between the molecular signature is determined between a reprogrammed somatic cell obtained from a diseased tissue or organ and a non-reprogrammed somatic cell obtained from a, or the, diseased tissue or organ. In a further embodiment, the difference between the molecular signature is determined between a reprogrammed somatic cell obtained from a diseased tissue or organ and a non-reprogrammed somatic cell obtained from a non-diseased tissue or organ. In an alternative embodiment, the difference between the molecular signature is determined between a reprogrammed somatic cell obtained from a non-diseased tissue or organ and a non-reprogrammed somatic cell obtained from a diseased tissue or organ. It will be appreciated that according to these embodiments, said diseased or non-diseased tissue or organ from which the reprogrammed somatic cell and/or non-reprogrammed somatic cell are obtained may be the same tissue or organ, such as a different part of the issue or organ, or from different tissues or organs.

### EXAMPLES

### Materials and Methods

Human fibroblasts from three different donors were simultaneously infected with lentiviruses containing the doxycycline-responsive transactivator (available from www.addgene.org) and the tetO-GFP-hOKMS construct (an inducible expression cassette encoding the Yamanaka factors as defined herein) in the presence of polybrene (8 μg/ml). Next, cells were centrifuged for 1 hour at 1000 rpm after the addition of the viruses to improve transduction efficiency. Doxycycline (2 μg/ml) was added to the fibroblast media (DMEM-F12, 10% FBS, 1×Glutamax, 1×MEM-NEAA, 1×β-ME, 0.2×Pen/Strep, 16 ng/ml FGF2) 24 hours after infection (day 0). Cells were then flow sorted (FACS) for GFP expression on day 2 of doxycycline treatment and re-plated onto gelatine-coated dishes. On day 7 post-infection, cells were passaged onto dishes containing irradiated mouse embryonic fibroblasts (iMEFs) and on the following day, the media was changed to human embryonic stem cell medium (DMEM-F12, 20% KSR, 1×Glutamax, 1×MEM-NEAA, 1×β-ME, 0.2×Pen/Strep, 8 ng/ml FGF2). On days 13, 15 and 17, cells undergoing reprogramming were flow sorted (FACS) for CD13 and SSEA4 surface expression using antibodies against those markers (obtained from Biolegend). Both the CD13+ SSEA4− and CD13− SSEA4+ populations were collected and re-plated in fibroblast medium onto dishes containing iMEFs. The re-plated cells were grown for four weeks without doxycycline so that they could revert to their initial cell type (“reversion” as defined herein). At the end of the four weeks of reversion, cells were harvested for flow cytometric analysis, DNA methylation array and RNA-sequencing.

### DNA Methylation Array

Genomic DNA was extracted from cell samples with the DNeasy blood and tissue kit (Qiagen) by following the manufacturer's instructions and including the optional RNase digestion step. Genomic DNA samples were processed further at the Barts and the London Genome Centre and run on an Infinium MethylationEPIC array (Illumina).

### RNA-Seq

RNA was extracted from cell samples with the RNeasy mini kit (Qiagen) by following the manufacturer's instructions. RNA samples were DNase treated (Thermo Scientific) to remove contaminating DNA. RNA-Seq libraries were prepared at the Wellcome Sanger Institute and run on a HiSeq 2500 system (Illumina) for 50 bp single-end sequencing.

### DNA Methylation Analysis

The array data was processed with the minfi R package and NOOB normalisation to generate beta values. DNA methylation age was calculated using the Horvath epigenetic clock (Horvath (2013) Genome Biology 14, R115). Reference datasets for reprogramming fibroblasts and iPSCs were obtained from Ohnuki et al (2014) Proc. Natl. Acad. Sci. 111, 12426-12431 and Banovich et al (2018) Genome Res 28, 122-131. In addition, the reference datasets included unpublished data examining the intermediate stages of fibroblasts being reprogrammed with the CytoTune™-iPS 2.0 Sendai Reprogramming kit (Invitrogen).

### RNA-Seq Analysis

Reads were trimmed with Trim Galore (version 0.6.2) and aligned to the human genome (GRCh38) with Hisat2 (version 2.1.0). Raw counts and log 2 transformed counts were generated with Seqmonk. Reference datasets for fibroblasts and iPSCs were obtained from Fleischer et al (2018) Genome Biol. 19, 221 and Banovich et al (2018) (supra). In addition, the reference datasets included unpublished data examining the intermediate stages of fibroblasts being reprogrammed with the CytoTune™-iPS 2.0 Sendai Reprogramming kit (Invitrogen).

Immunofluorescence and Imaging Antibody staining was performed as previously described (Santos et al (2003) Curr. Biol. 13, 1116-1121) on cells grown on coverslips or cytospun, after fixation with 2% PFA for 30 minutes at room temperature. Briefly, cells were permeabilised with 0.5% TritonX-100 in PBS for 1 h; blocked with 1% BSA in 0.05% Tween20 in PBS (BS) for 1 h; incubation of the appropriate primary antibody diluted in BS; followed by wash in BS and secondary antibody. All secondary antibodies were Alexa Fluor conjugated (Molecular Probes) diluted 1:1000 in BS and incubated for 30 minutes. Incubations were performed at room temperature. DNA was counterstained with 5 μg/mL DAPI in PBS. Optical sections were captured with a Zeiss LSM780 microscope (63× oil-immersion objective). Fluorescence semi-quantification analysis was performed with Volocity 6.3 (Improvision). Antibodies used are listed below:

### Anti-H3K9me3; 07-442, Merck/Millipore (1:500);

Anti-Collagen I; ab254113, Abcam (1:400).

### Example 1: Partially Reprogrammed Somatic Cells are Fibroblast-Like

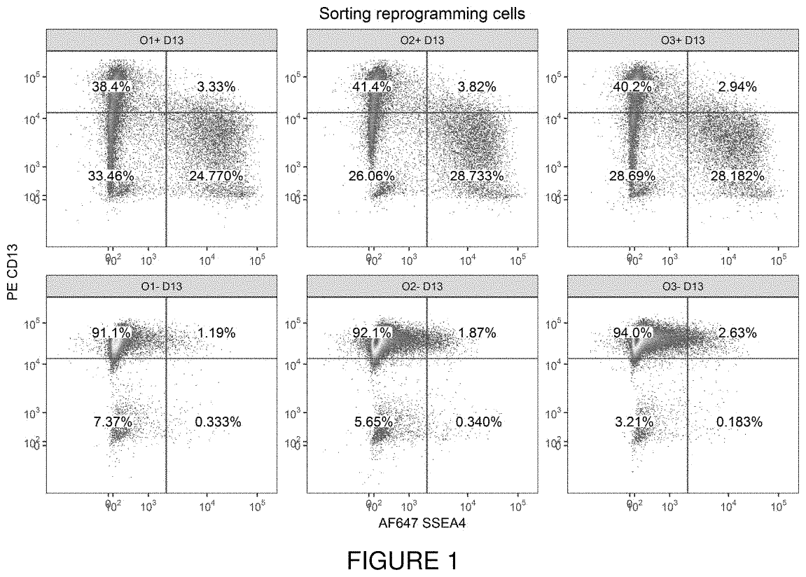

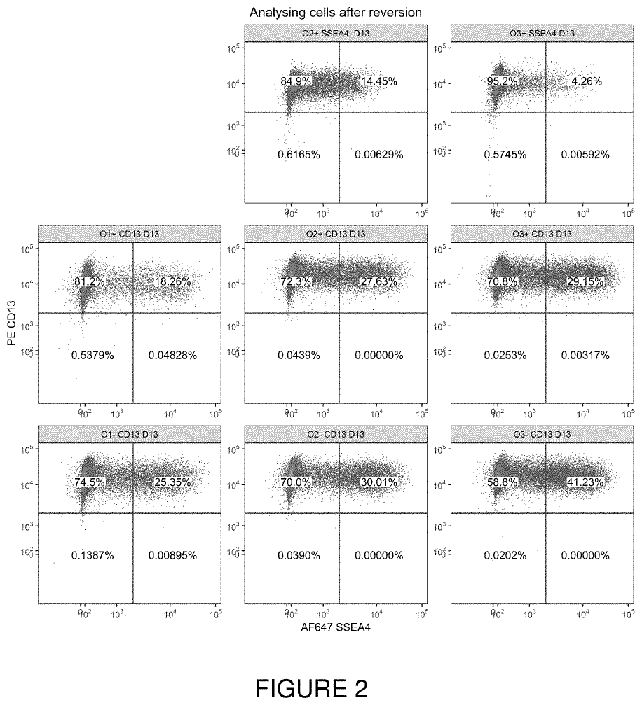

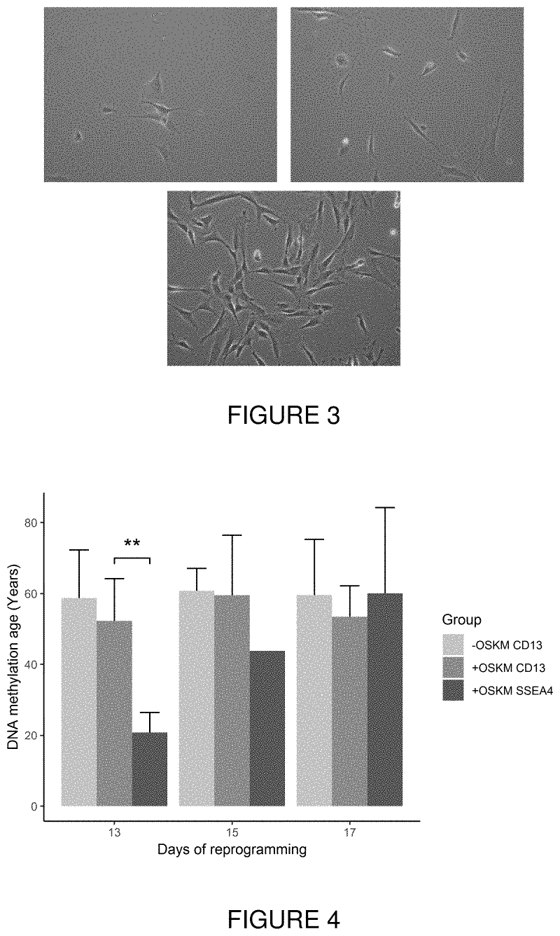

At the intermediate stages of the protocol, cells that were expressing the Yamanaka factors became heterogeneous. Some cells remained CD13+ SSEA4− and were classed as non-reprogramming, while some cells became CD13− SSEA4+ and were classed as successfully reprogramming (see FIG. 1). Both of these populations were flow sorted (FACS) and cultured for 4 additional weeks without doxycycline (“reversion” as defined herein). At the end of this period, the successfully reprogramming cells returned to a fibroblast phenotype and became CD13+ SSEA4− (see FIG. 2). These cells also resembled fibroblasts morphologically (see FIG. 3). Cells that were non-reprogramming (shown in plots labelled “+CD13”) or that were not cultured in the presence of the Yamanaka factors (“−” conditions) remained fibroblast-like throughout.

Similar findings were found for longer periods of reprogramming (data not shown).

### Example 2: Partially Reprogrammed Somatic Cells Display a Younger Epigenetic Age

Cells that were successfully reprogramming after 13 days of culture in the presence of Yamanaka factors and then reverted displayed a DNA methylation age of 30-40 years younger than their respective controls. Carrying out longer periods of reprogramming (15 or 17 days) before the reversion phase slightly reduced the rejuvenation effect. Non-reprogramming cells that had expressed the Yamanaka factors were the same age as the negative controls that had never expressed the Yamanaka factors (see FIG. 4).

The data presented herein therefore shows that expressing the Yamanaka factors alone for short periods of time (i.e. for a period prior to expression of a pluripotency marker, such as SSEA4, prior to when a somatic cell lineage-specific marker expression is no longer detectable on the cell surface, within the initiation phase of iPS cell reprogramming, or less than 5 days) is not sufficient to rejuvenate the epigenetic age or to successfully reprogram somatic cells to display a younger DNA methylation age/epigenetic signature. The data also suggests that, in order to be considered successfully reprogramming, the cells must also become positive for the reprogramming/pluripotency marker SSEA4.

### Example 3: Transient Reprogramming Experiments

### (a) Experimental Design

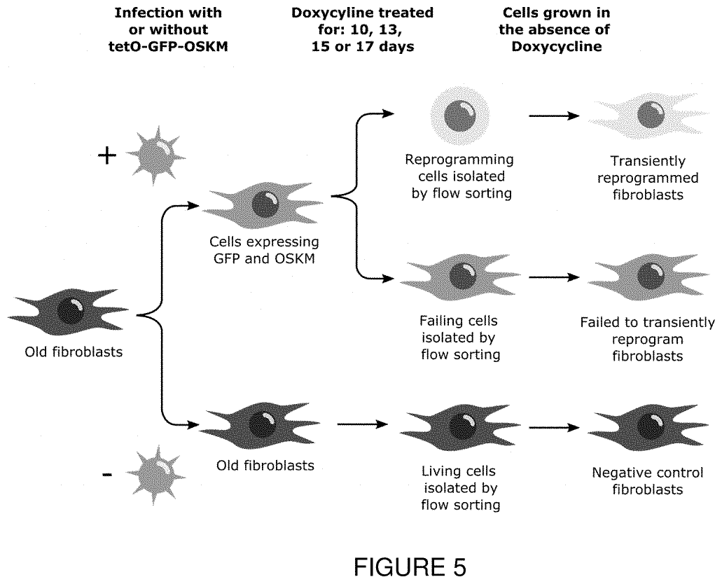

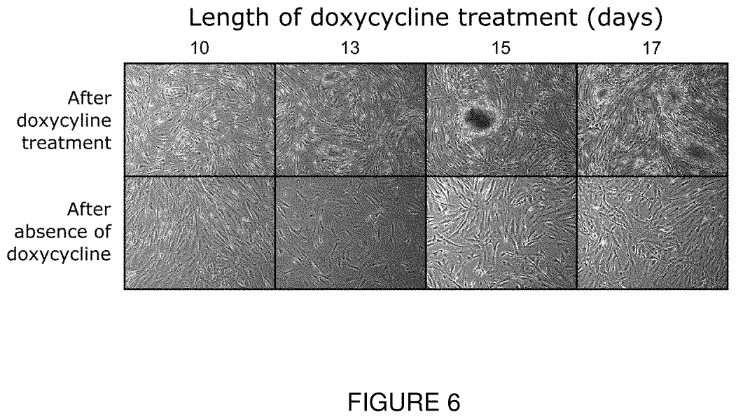

To investigate the potential of transient reprogramming, we infected fibroblasts from old donors with a doxycycline-inducible reprogramming construct, containing Oct4, Sox2, Klf4, c-Myc and GFP. In addition, we ‘mock infected’ some fibroblasts without using the construct to generate the negative control. After infection, we positively selected the GFP expressing cells with flow sorting and for the negative control, we flow sorted equal numbers of viable cells. We then treated the cells with doxycycline for different lengths of time before flow sorting for successfully reprogramming cells and cells failing to reprogram based on the cell surface markers: CD13 and SSEA4. Finally, we grew the sorted cells in the absence of doxycycline for a period of at least 4 weeks before collecting the cells for analysis (FIG. 5). Cells morphologically resembled fibroblasts at the end of the process (FIG. 6).

### (b) Transiently Reprogrammed Cells Epigenetically Resemble Fibroblasts

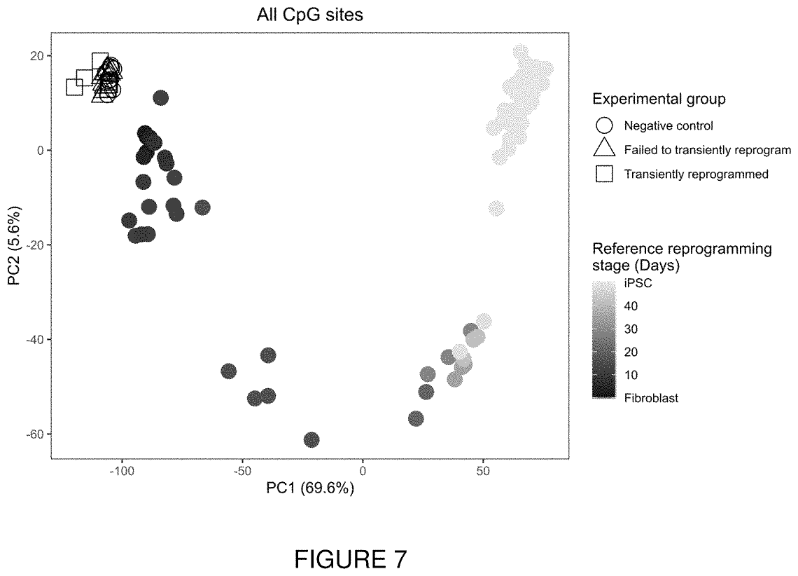

We carried out principal component analysis on the methylomes of transiently reprogrammed cells alongside reference datasets examining complete fibroblast reprogramming. As expected, principal component 1 separated cells based on extent of reprogramming and generated a reprogramming trajectory with the reference datasets. Notably, the transiently reprogrammed cells (and the control groups) resided at the start of the reprogramming trajectory suggesting they resemble fibroblasts rather than reprogramming intermediates or iPSCs (FIG. 7).

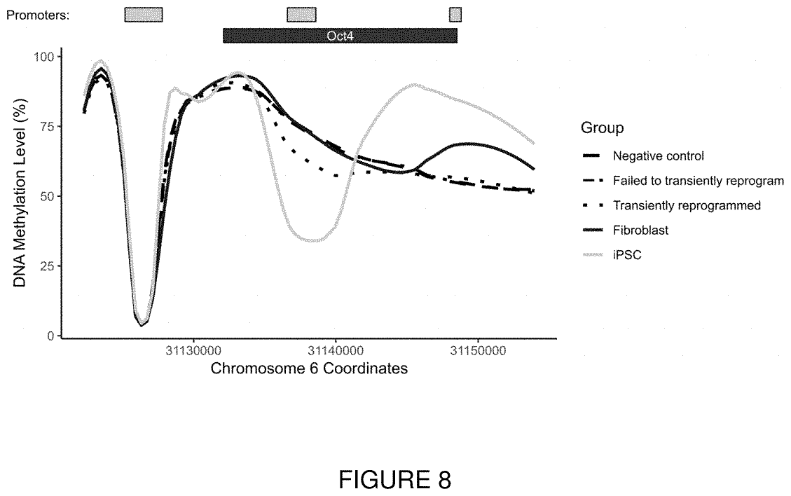

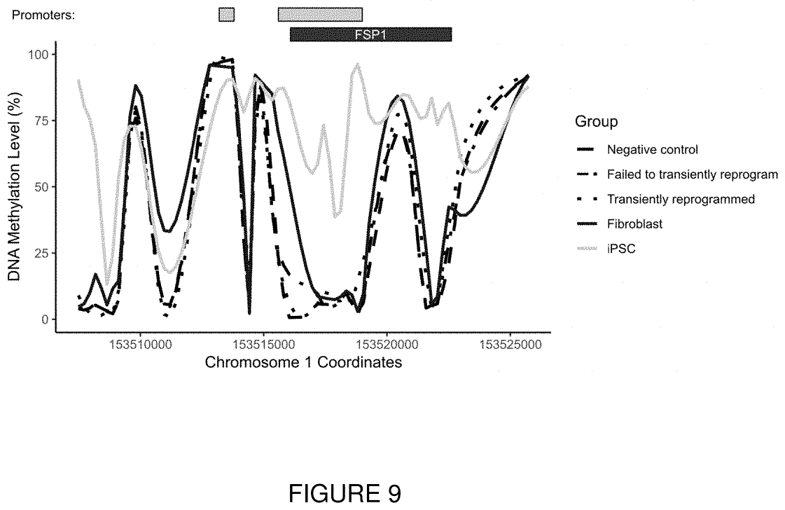

During iPSC reprogramming, many DNA methylation changes occur at regulatory elements such as promoters. Notably the Oct4 promoter becomes demethylated during iPSC reprogramming. However, when we examined the Oct4 promoter in the transiently reprogrammed cells, we found that it was still hypermethylated at levels similar to the control groups and reference fibroblasts (FIG. 8). We also observed that the promoter of FSP1 (a fibroblast marker gene) becomes hypermethylated in iPSCs. Though in transiently reprogrammed cells, we found this promoter was still demethylated (FIG. 9).

### (c) Transiently Reprogrammed Cells Transcriptionally Resemble Fibroblasts

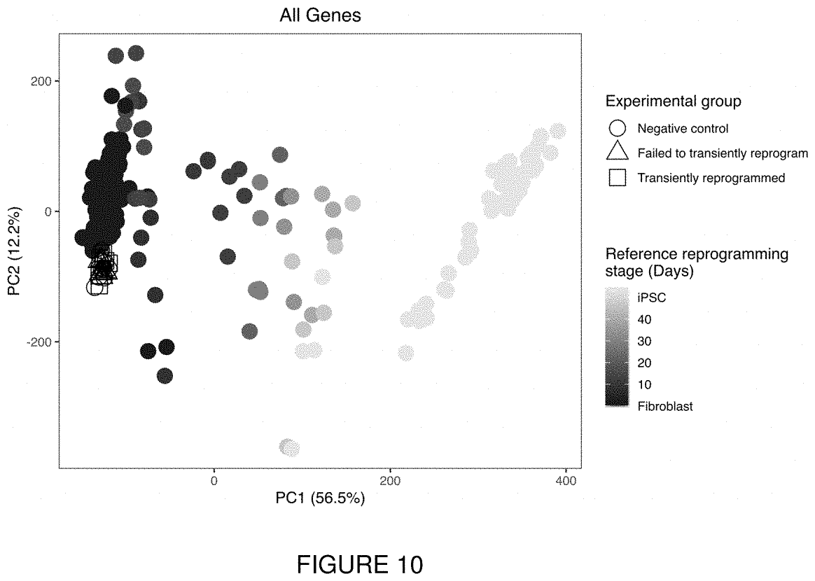

We also carried out principal component analysis on the transcriptomes of transiently reprogrammed cells together with reference datasets examining complete fibroblast reprogramming. Like the methylome analysis, principal component 1 separated samples based on extent of reprogramming and generated a reprogramming trajectory with the reference datasets. The transcriptomes of transiently reprogrammed cells resided at the beginning of this trajectory suggesting that these cells also transcriptionally resemble fibroblasts (FIG. 10).

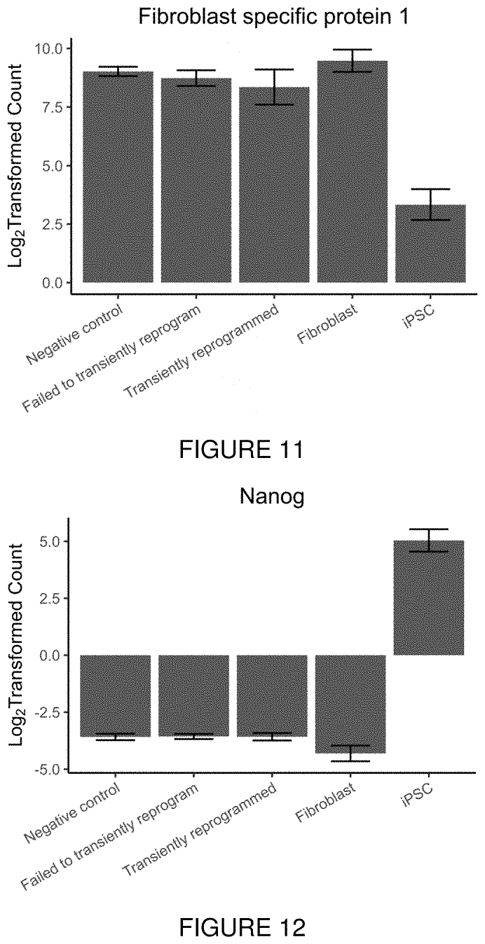

During iPSC reprogramming, marker genes of the starting cell type are downregulated, and pluripotency genes are upregulated. We found that fibroblast marker genes such as FSP1 (FIG. 11) were not downregulated in transiently reprogrammed cells and pluripotency marker genes such as Nanog (FIG. 12) were not upregulated.

### (d) Transient Reprogramming Rejuvenates Many Markers of Ageing

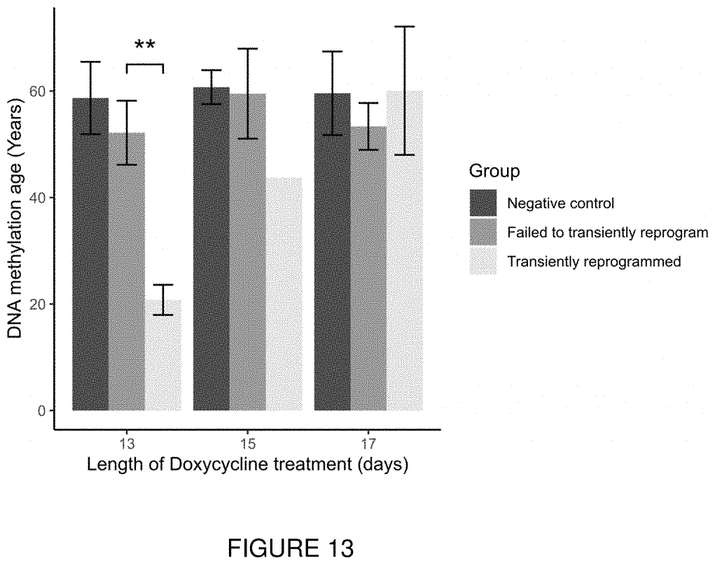

To investigate the rejuvenating effects of transient reprogramming on the epigenome, we calculated the DNA methylation age of cells after transient reprogramming with the Horvath epigenetic clock. We found that transient reprogramming rejuvenated DNA methylation by up to 40 years relative to the control groups. Notably, transient reprogramming with 13-day doxycycline treatment resulted in the most rejuvenation suggesting this is the optimal amount for epigenetic rejuvenation (FIG. 13).

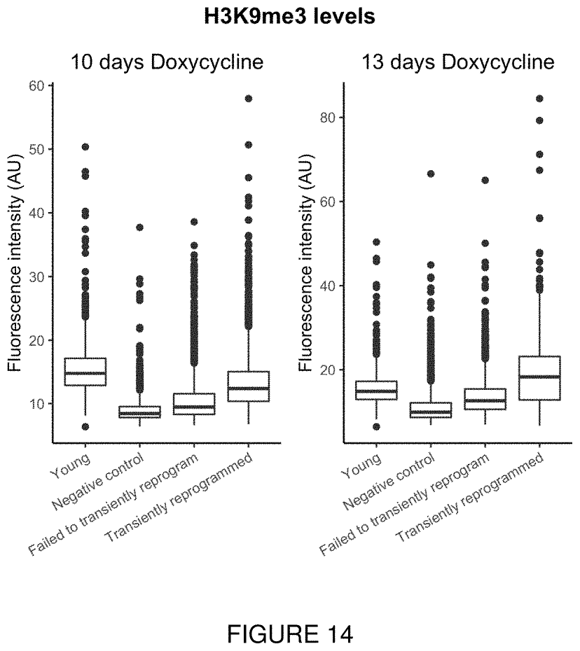

Other features of the epigenome change with ageing, such as global levels of histone modifications. H3K9me3 levels decrease with ageing and we found that transient reprogramming has the potential to increase H3K9me3 to youthful levels (FIG. 14).

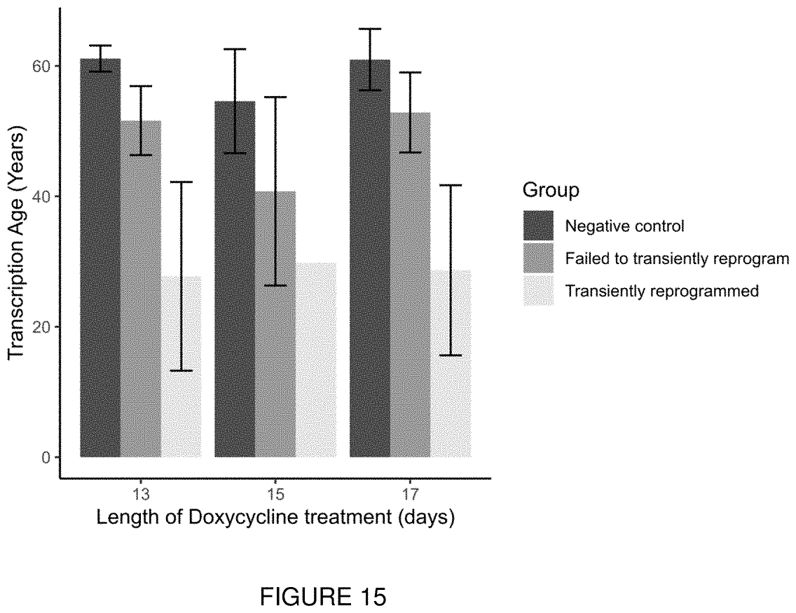

To investigate the rejuvenating effects of transient reprogramming on the transcriptome, we trained a transcription clock using random forest regression on published data from fibroblasts (Fleischer et al (2018), supra). This transcription clock predicted age with a median absolute error of 13.48 years. Using this clock, we found that transient reprogramming rejuvenated transcription age by approximately 30-40 years, which was similar to the extent of rejuvenation observed with the epigenetic clock. Contrary to the epigenetic clock, transcription age rejuvenation was observed for all lengths of doxycycline treatment investigated (FIG. 15).

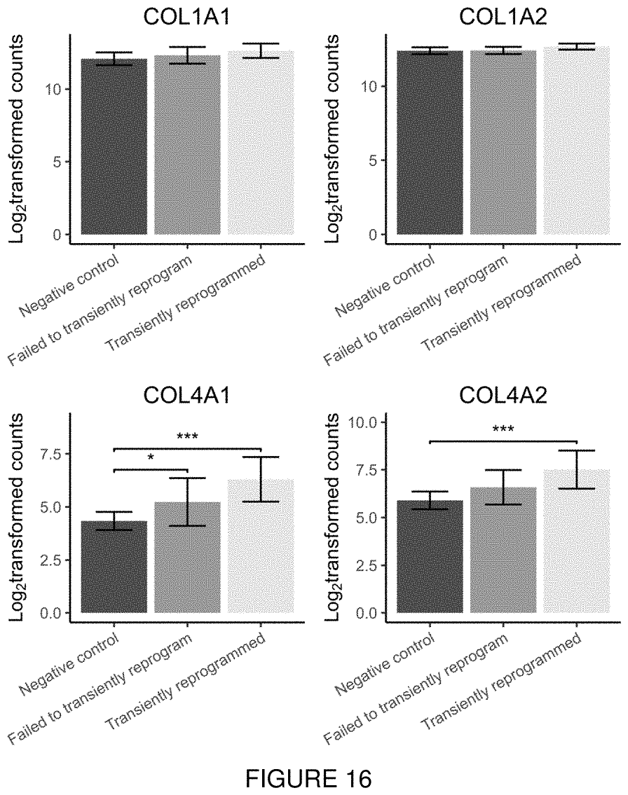

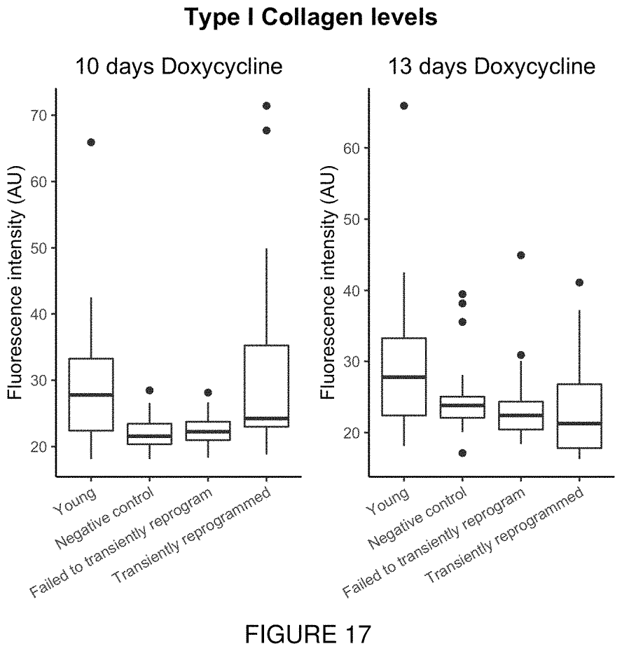

Secretion of collagen is a key function of fibroblasts. We found that transient reprogramming increased the expression of several collagen genes. Notably these increases were highly significant for COL4A1 and COL4A2 (FIG. 16). We also investigated the protein levels of type I collagen by immunofluorescence and found that transient reprogramming (with a 10-day Doxycycline treatment) restored collagen protein to youthful levels (FIG. 17).

1. A method of reprogramming a somatic cell to a pluripotent-like or rejuvenated state, comprising:
i) culturing said somatic cell in the presence of one or more Yamanaka factors for a period of at least 5 days, and/or until expression of a pluripotency marker is detectable on the surface of or within said somatic cell, and/or until a somatic cell lineage-specific marker is no longer detectable on the surface of said somatic cell;
ii) further culturing said somatic cell in the absence of said one or more Yamanaka factors until expression of said pluripotency marker has reduced on the surface of or within said somatic cell, and/or until expression of a somatic cell lineage-specific marker is detected on the surface of said somatic cell.

2. The method of claim 1, wherein said method reprograms a somatic cell to a rejuvenated state.

3. The method of claim 1, wherein the somatic cell is cultured in the presence of said one or more Yamanaka factors for a period of at least 6 days, or at least 13 days.

4. The method of any one of claims 1 to 3, wherein the somatic cell is cultured in the presence of said one or more Yamanaka factors for a period of no more than 17 days, or no more than 15 days.

5. The method of any one of claims 1 to 4, wherein the somatic cell is cultured in the presence of said one or more Yamanaka factors until expression of a pluripotency marker is detectable on the surface of or in said somatic cell.

6. The method of any one of claims 1 to 5, wherein the pluripotency marker is stage specific embryonic antigen-4 (SSEA4).

7. The method of any one of claims 1 to 6, wherein the somatic cell is cultured in the presence of said one or more Yamanaka factors until expression of a somatic cell lineage-specific marker is no longer detected on the surface of said somatic cell.

8. The method of any one of claims 1 to 7, wherein the somatic cell is cultured in the absence of said one or more Yamanaka factors until expression of said pluripotency marker is reduced on the surface of said somatic cell.

9. The method of any one of claims 1 to 8, wherein the somatic cell is cultured in the absence of said one or more Yamanaka factors until expression of a somatic cell lineage-specific marker is detected on the surface of said somatic cell.

10. The method of any one of claims 1 to 9, wherein the reprogramming of the somatic cell is incomplete and/or partial reprogramming and/or transient reprogramming.

11. The method of any one of claims 1 to 10, wherein the somatic cell is cultured in the presence of said one or more Yamanaka factors within the maturation phase of reprogramming.

12. The method of any one of claims 1 to 11, wherein the reprogrammed somatic cell comprises a molecular signature, such as an epigenetic signature, corresponding to a somatic cell from an earlier point in the life cycle of the tissue.

13. The method of any one of claims 1 to 12, wherein the reprogrammed somatic cell retains the phenotype and/or the molecular signature, such as the epigenetic signature, of the non-reprogrammed somatic cell.

14. The method of any one of claims 1 to 13, wherein the molecular signature of the reprogrammed and/or non-reprogrammed somatic cell is the epigenetic signature and is determined using the Horvath epigenetic clock, such as wherein the epigenetic signature of the reprogrammed somatic cell indicates an epigenetic age of at least 10% younger, at least 40% younger, or at least 70% younger than the non-reprogrammed somatic cell.

15. The method of any one of claims 1 to 14, wherein the one or more Yamanaka factors are provided from an inducible expression cassette transduced or transfected into the somatic cell, such as wherein the culturing in the presence of said one or more Yamanaka factors further comprises addition of a compound capable of inducing expression from the inducible expression cassette.

16. The method of any one of claims 1 to 15, wherein the culturing in the absence of said one or more Yamanaka factors comprises removal of the compound capable of inducing expression from the inducible expression cassette.

17. The method of any one of claims 1 to 16, wherein the one or more Yamanaka factors are provided in the form of Yamanaka factor-encoding mRNA.

18. The method of claim 17, wherein the culturing in the absence of said one or more Yamanaka factors comprises removal of the Yamanaka factor-encoding mRNA from culture.

19. The method of any one of claims 1 to 16, wherein the one or more Yamanaka factors are provided in the form of proteins expressed from Yamanaka factor-encoding mRNA.

20. The method of claim 19, wherein the proteins are directly delivered to the somatic cell by targeted delivery systems, such as functional twin-arginine translocation (Tat) systems or nanoparticle delivery systems.

21. The method of any one of claims 1 to 16, wherein the Yamanaka factors are introduced into the somatic cell by CRISPR/Cas-9, such as drug- (i.e. doxycycline (dox)) inducible or non-inducible CRISPR/Cas-9.

22. The method of any one of claims 1 to 21, wherein the one or more Yamanaka factors is selected from one or more of: OCT4, KLF4, c-MYC, SOX2, LIN28 and/or NANOG, or is preferably selected from one or more, or two or more, or three or more, or all of: OCT4, KLF4, c-MYC and/or SOX2.

23. A reprogrammed somatic cell produced according to the method of any one of claims 1 to 22.

24. A pharmaceutical composition comprising a reprogrammed somatic cell according to claim 23.

25. The reprogrammed somatic cell according to claim 23 or the pharmaceutical composition according to claim 24 for use in the treatment and/or amelioration of a degenerative or age-related disease or disorder or for use in the rejuvenation of a tissue or organ, such as skin, blood, bone marrow, liver or heart, wherein the degenerative or age-related disease or disorder comprises: a disease or disorder of the skin; or a disease or disorder of the pancreas, e.g. type 2 diabetes; or a neurodegenerative disorder.

26. The reprogrammed somatic cell according to claim 23 or the pharmaceutical composition according to claim 24 for use of claim 25, which is for administration to a human or animal subject.

27. A cosmetic composition comprising a reprogrammed somatic cell according to claim 23.

28. A cosmetic method of regenerating or rejuvenating skin comprising administration or application of a reprogrammed somatic cell according to claim 23 or the cosmetic composition of claim 27 to a subject in need thereof.

29. A method of screening for an age modulating agent, said method comprising:
(i) performing the method of any one of claims 1 to 22 in the presence and the absence of a test agent to generate a reprogrammed somatic cell; and
(ii) determining the molecular signature, such as the epigenetic signature, of the reprogrammed somatic cell,
wherein a difference between the molecular signature determined for a reprogrammed somatic cell generated in the presence of the test agent and the molecular signature determined for a reprogrammed somatic cell generated in the absence of the test agent is indicative of the age modulating effect of said agent.

30. A method of screening for an age modulating factor or cellular process, said method comprising:
(i) reprogramming a somatic cell from a diseased tissue or organ according to the method of any one of claims 1 to 22; and
(ii) determining the molecular signature, such as the epigenetic signature, of the reprogrammed somatic cell from a diseased tissue or organ and of a reprogrammed somatic cell of claim 23 or of a non-reprogrammed somatic cell from said diseased tissue or organ,
wherein a difference between the molecular signature determined for the reprogrammed somatic cell from a diseased tissue or organ and the molecular signature determined for the reprogrammed somatic cell of claim 23 or the non-reprogrammed somatic cell from the diseased tissue or organ is indicative of the age modulating factor or cellular process associated with the disease.
